(19) United States
(12) Patent Application Publication
(10) Pub. No.: US 2007/0159813 A1
(43) Pub. Date: Jul. 12, 2007

(54) Illuminated artificial flowers and manufacturing and assembly methods thereof

(76) Inventor: Joseph Whittle Husbands, Wedowee, AL (US)

Correspondence Address: CAHN & SAMUELS LLP, 2000 P STREET NW, SUITE 200, WASHINGTON, DC, 20036, US

(21) Appl. No.: 11/371,011

(22) Filed: Mar. 9, 2006

Related U.S. Application Data
(60) Provisional application No. 60/757,038, filed on Jan. 9, 2006.

(51) Int. Cl. F21S 6/00; A01H 5/00

(52) U.S. Cl. 362/122; PLT/139

(57) ABSTRACT

An artificial flower assembly and manufacturing and assembly methods are disclosed including a stem made of a flexible tubular shaft, an electrical socket, and a collar surrounding the socket and connected to the upper end of the shaft; a removable blossom with an opening; and a bulb threaded through the blossom opening and engaged with the socket, a portion of the bulb abutting the blossom and holding the blossom onto the stem.

### I. RELATED APPLICATIONS

This application is an application filed under 35 U.S.C. §111(a) claiming benefit pursuant to 35 U.S.C. §119(e)(1) of the filing date of the Provisional Application 60/757,038 filed on Jan. 9, 2006 pursuant to 35 U.S.C. §111(b), the entire contents of which are incorporated herein by reference.

### II. FIELD OF THE INVENTION

This invention relates to improvements to the traditional illuminated artificial flower and manufacturing and assembly methods thereof.

### III. BACKGROUND OF THE INVENTION

Traditionally, artificial flowers are made of materials, such as silk, paper, or fabric that, while having a realistic appearance, have limited durability. Savings in cost and efficiency are gained if more durable materials are used.

The materials used to construct artificial flower arrangements are also typically of a fixed appearance, not readily subject to change.

Artificial flowers illuminated with a bulb or LED typically consist of complicated structural and electrical components. The materials and processes used to attach petals and light sockets to various artificial flower stems require substantial custom manufacturing and assembly. Reductions in time, labor and cost can be achieved if simpler materials and assembly processes are used.

Artificial flowers placed outdoors using unaltered line voltage power typically require a covering and watertight seal over the light bulb for safety reasons. This results in different designs for indoor and outdoor applications. Alternatively, a transformer might be used to lower the voltage. However, attachment of the transformer to the outside of the artificial flower assembly can be cumbersome.

Outdoor landscaping uses of artificial flowers require a means to support the flower stem in the ground. Typically, this is accomplished with stakes having cumbersome designs for attachment to the outside of the flower stem.

Notwithstanding the usefulness of the above-described structures typically used with illuminated artificial flowers, a need still exists for an illuminated artificial flower made of durable materials, manufactured and assembled in a convenient and efficient manner, capable of changing appearance due to oxidation or in response to changes in temperature, moisture, or light, with a transformer contained within the assembly and a landscaping stake easily fitting inside the flower stem.

### IV. SUMMARY OF THE INVENTION

It is an object of at least one embodiment of the invention to overcome the foregoing exemplary shortcomings and problems.

It is another object of at least one embodiment of the invention to provide an illuminated artificial flower assembly manufactured from durable materials and capable of being used both indoors and outdoors. More durable materials, for example, thin sheet metals such as aluminum or copper not only extend the life of the artificial flower but also allow the flower to be readily used outdoors (on a patio or in landscaping arrangements, for example). Use of such materials also allows attachment of appendages, such as leaves, by convenient methods, for example, riveting. Also, an artificial flower assembly can be designed for outdoor applications using a transformer lowering the voltage of the electric current. With a transformer, the bulb can safely remain uncovered and unsealed, thereby allowing the same stem and bulb socket assembly to be used in indoors and outdoors. In the past, such transformers have been located external to the artificial flower assembly. Incorporation of the transformer into the flower assembly increases protection provided to the electrical components and also makes for easier carrying and placement of the assembly.

It is a further object of at least one embodiment of the invention to provide an illuminated artificial flower assembly capable of being manufactured and assembled conveniently and at a reduced cost, using materials that are easily obtained. For example, a durable colored heat-shrink plastic sleeve, typically used to bundle a plurality of wires, can also be used to secure electrical components, such as the bulb socket, to the flower stem. Also, common plumbing fixtures, which might create an awkward appearance if used with silk, fabric or paper, can be used with aesthetic appeal in artificial flowers made from sheet metals.

Another object of at least one embodiment of the invention is to provide an efficient and easy stake for supporting the artificial flower assembly stem to the ground in landscaping applications. Efficiencies in manufacturing, cost and application are achieved when the flower stem is supported by way of a readily available, generally rigid rod, one end inserted into the ground and the other end fitting neatly inside the base of the stem.

It is also an object of at least one embodiment of the invention to provide an artificial flower assembly constructed of materials such as copper, phosphorescent, or other materials which change color and/or appearance over time, as a result of exposure to light, or due to changes in temperature or moisture/rainfall. Copper, for example, changes appearance as a result of oxidation from a shiny bright orange surface to a dull grey-green patina. Enjoyment of the artificial flower is enhanced if the stem, leaves, or petals/blossom of the flower are made of such materials.

It is still a further object of at least one embodiment of this invention to provide an artificial flower and methods for assembling the same whose parts are easily removable and replaced. Additional enjoyment of the artificial flower is gained if certain portions, the petals/blossom for example, are easily changed, thereby allowing the same stem to be used for numerous multiple events. For example, petals of green, red and white might be used for Christmas, black and orange for Halloween, pink or blue for new babies, petals/blossom bearing the particular colors of a favorite sporting team.

In one embodiment of the invention an illuminated artificial flower assembly includes a stem comprising a flexible tubular shaft, an electrical socket, and a collar. The collar surrounds the electrical socket and is connected to the upper end of the shaft. The embodiment also includes a removable blossom having an opening of a first diameter. The embodiment includes a bulb with a base, and a portion of the base is sized larger than the blossom opening. The bulb is threaded through the blossom opening and engaged with the socket. The portion of the bulb base that is larger than the blossom opening holds the blossom securely in place on the stem.

In yet another embodiment, the flower assembly also includes a leaf attached to the stem. The shaft, the collar, and the blossom are made of copper. The embodiment also includes a container with a compartment holding the stem and a transformer, the transformer being electrically connected to the stem.

Another embodiment comprises a kit for the flower assembly. The kit includes a plurality of stems comprising a flexible tubular shaft, an electrical socket, and a collar surrounding the electrical socket and connected to the upper end of the shaft. The embodiment also includes a plurality of blossoms, each having an opening of a first diameter, and a plurality of bulbs adapted to thread through the blossom opening and engage with the socket and a portion of the bulb being adapted to abut the blossom and secure the blossom onto the stem. The kit also includes a plurality of stakes each adapted to associate with the lower end of the shaft and support the flower assembly.

A further embodiment of the invention defines a method for manufacturing the artificial flower assembly. The method includes associating the socket wire with the shaft, surrounding the socket with the collar, and connecting the collar to the upper end of the shaft.

Still another embodiment of the invention defines a method for assembling the artificial flower assembly. The method includes threading the bulb through the opening of the blossom and engaging the bulb with the socket, thereby securing the blossom to the stem.

In the following enabling description, reference is made to the accompanying drawings which are shown by way of illustration of the specific embodiments in which the invention may be practiced. In the following embodiments the apparatus and methods should become evident to a person of ordinary skill in the art and in sufficient detail to enable those skilled in the art to practice the invention. It is to be understood that other embodiments may be used and that structural changes based on presently known structural and/or functional equivalents may be made without departing from the scope of the invention.

### V. BRIEF DESCRIPTION OF THE DRAWINGS

FIG. 1 illustrates an assembled view of an embodiment of the artificial flower assembly with a plurality of leafs.

FIG. 2 depicts a perspective view of the embodiment of FIG. 1.

FIG. 3 depicts an assembly view of an embodiment of the flower assembly with a collar comprising a reducer fitting.

FIG. 4 illustrates the collar of the embodiment illustrated in FIG. 3 attached to the shaft.

FIG. 5 depicts a collar integrally attached to the shaft.

FIG. 6 depicts an assembly view of an embodiment of the flower assembly with a collar comprising a reducer fitting and a coupling.

FIG. 7A depicts an assembly view of the collar of the embodiment illustrated in FIG. 6.

FIG. 7B illustrates the collar of the embodiment illustrated in FIG. 6 attached to the shaft.

FIG. 8 depicts an assembly view of an embodiment of the flower assembly with a collar comprising a heat-shrinking sleeve.

FIG. 9 illustrates the assembled collar of the embodiment illustrated in FIG. 8 attached to the shaft.

FIG. 10 illustrates the assembled embodiment illustrated in FIG. 8.

FIG. 11A depicts an assembly view of an embodiment of the flower assembly with a collar comprising a heat-shrinking sleeve and two spacers between the blossom and the socket.

FIG. 11B depicts an assembly view of an embodiment of the flower assembly with a collar comprising a heat-shrinking sleeve and the socket having screw threads.

FIG. 12 illustrates the assembled embodiment illustrated in FIG. 11A.

FIG. 13 illustrates an assembly view of an embodiment of the artificial flower assembly with the stem secured to the platform and the container including a transformer.

FIG. 14 depicts a plurality of stems disposed in a container.

FIG. 15 illustrates an assembly view of an embodiment of the flower assembly with a rod stake.

FIG. 16 illustrates an assembled view of an embodiment with a timer.

FIG. 17 illustrates an assembled view of an embodiment with a solar power device.

### VI. DETAILED DESCRIPTION OF THE INVENTION

As used herein, “substantially,” “generally,” “relatively” and other words of degree are relative modifiers intended to indicate permissible variation from the characteristic so modified. They are not intended to be limited to the absolute value or characteristic which they modify, but rather possessing more of the physical or functional characteristic than its opposite, and preferably approaching or approximating such a physical or functional characteristic.

As used herein, the phrase “blossom” refers to the portion of the artificial flower assembly between the bulb and the stem resembling one or a plurality of petals, blooms, corolla, perianth, sepal, calyx, leaves or other botanical ornamentation typically found at the end of a branch, stalk, or stem.

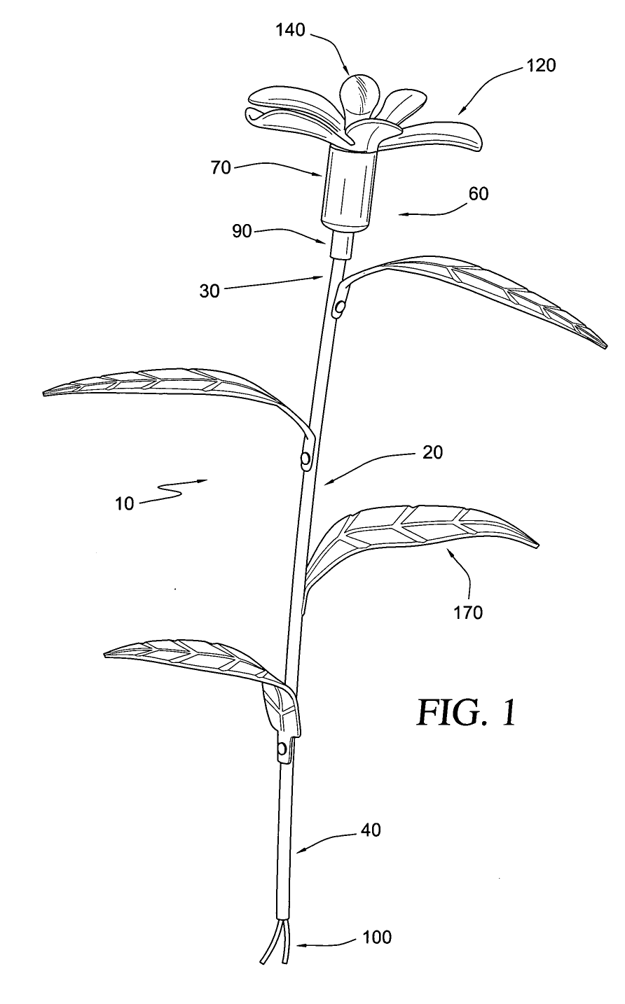

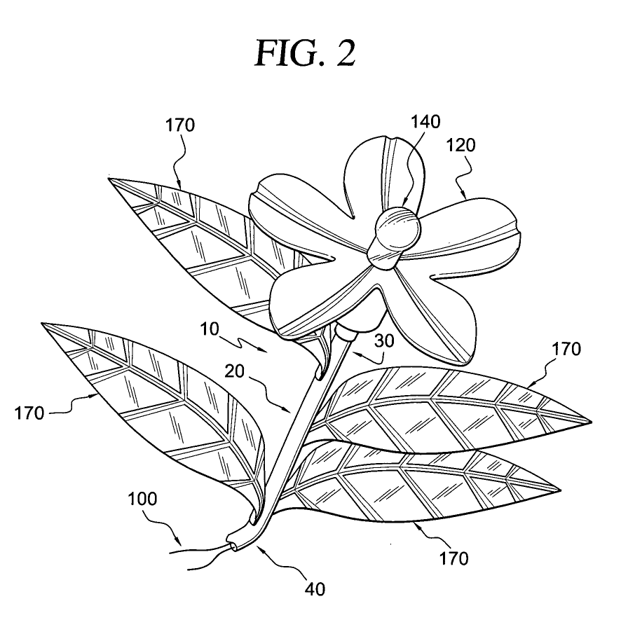

The present invention is generally directed to illuminated artificial flowers and methods of manufacturing and assembling the same. An artificial flower in accordance with one embodiment, illustrated in FIGS. 1 and 2, comprises a stem 10 including a shaft 20 made of a flexible material thereby enabling it to be positioned for maximum visual effect. The shaft 20 has an upper end 30 and lower end 40. An electrical socket 50 is protected by a collar 60 that is attached to the upper end 30 of the shaft 20. In some embodiments the collar 60 helps secure the electrical socket 50 to the upper end of the shaft 20. The artificial flower assembly also includes a bulb 140 and a removable blossom 120 sandwiched between the bulb 140 and the electrical socket 50.

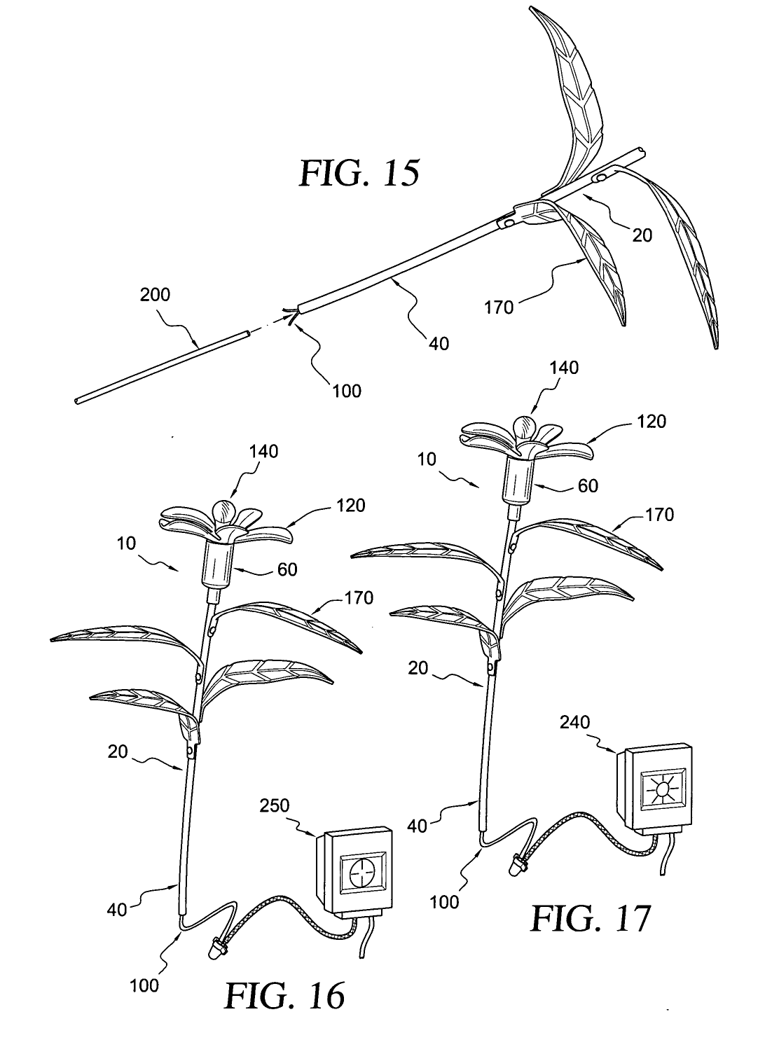

In the embodiments illustrated in FIGS. 1 and 2, a plurality of leafs 170 are attached to the shaft 20 of the stem 10. The leaf 170 can be attached, for example, by riveting. (See FIGS. 1 and 15).

Parts of the flower assembly, including but not limited to the blossoms 120, leafs 170, shaft 20 and collar 60, can be in various forms, resembling both known and fictional flowers or plants. Parts of the flower can also be in a variety of colors (for example, by spray-painting or colored aluminum or plastic). For example, an aluminum leaf 170, a plastic shaft 20, and a shrink-wrap collar might be colored green and a blossom 120 spray-painted red. The color combinations can be random or can have special significance (such as pink or blue for the birth of a baby, or the colors of a favorite football team). Because the blossoms 120 are removable and interchangeable, the same stem can be used by the purchaser for multiple events.

Parts of the artificial flower assembly, including but not limited to the blossoms 120, leafs 170, shaft 20 and collar 60, can also be made of a variety of materials. In one embodiment, the blossom 120 and the leaf 170 are made of aluminum and the collar 60 is a shrink-wrap plastic. In another embodiment, the blossom 120, the leaf 170, the shaft 20, and the collar 60 are made of copper. The use of such materials allows the parts of the flower assembly to be bent into convenient positions for shipping and to also be bent by the user into a variety of positions and forms, for example, resembling the appearance of plants in nature. Oxidation causes copper to change from a shiny bright orange surface to a blue-green patina. This is a change of both color and surface texture. The use of copper as a material, therefore, allows the natural process of oxidation to change the appearance of the copper parts of the artificial flower assembly over time. According to the invention, the oxidation process can be enhanced by applying a solution to the flower assembly. For example, Patina Blue Green Verde #445 can be applied to the copper parts to accelerate the oxidation process.

Parts of the artificial flower assembly, including but not limited to the blossoms 120, leafs 170, shaft 20 and collar 60,can also be made of other materials that change appearance or color due to oxidation, changes in temperature or moisture, or can be made of phosphorescent materials, causing any one or a combination of the artificial flower assembly parts to glow in the dark.

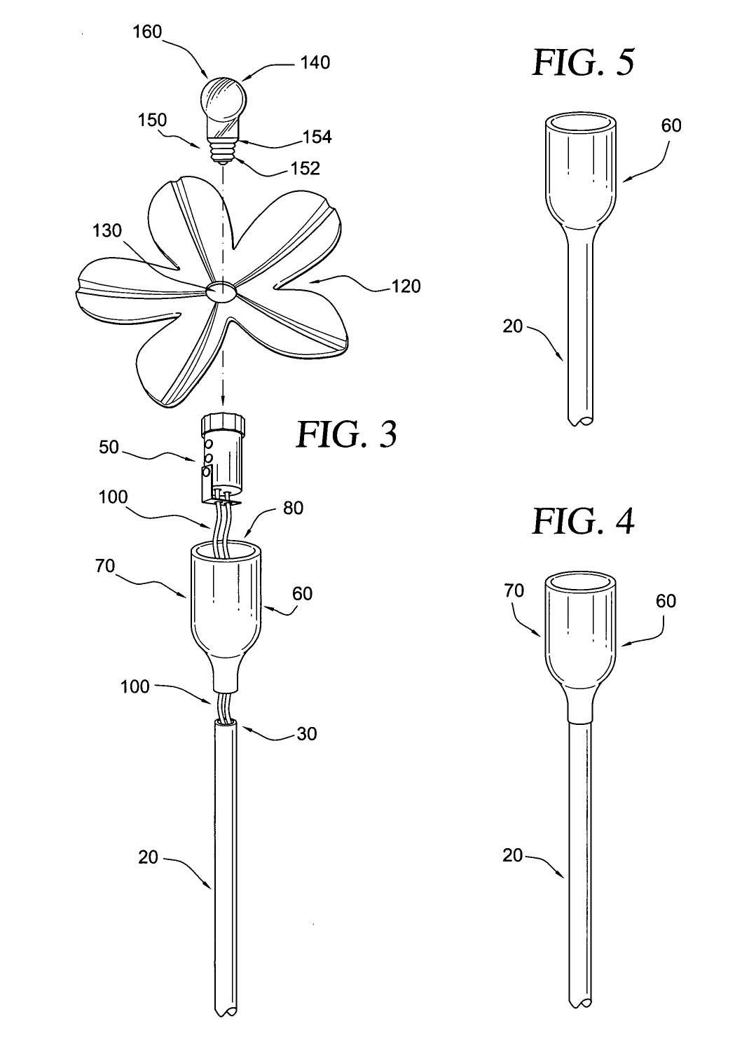

In accordance with the present invention, the blossom 120 may be removable. The blossom 120 has an opening 130 and is secured to the stem 10 by threading the bulb 140 through the opening 130 and engaging a portion of the bulb 140 with the socket 50. FIGS. 1, 2, 10, and 12, illustrate the blossom 120 and bulb 140 attached to the stem 10. The blossom 120 can be removed from the stem 10 by disengaging the bulb 140 from the socket 50. In the embodiments illustrated in FIGS. 3, 6, 8 and 11A, the bulb 140 engages the socket 50 by screw tightening. The bulb 140 can also engage the socket 50 by other means.

The blossom 120 is held in place on the stem 10 by the portion of the bulb 140 that does not pass through the opening 130 of the blossom 120. This portion of the bulb 140 abuts the blossom 120, holding the blossom 120 secure, while the portion of the bulb 140 passing through the opening 130 in the blossom 120 engages the socket 50.

In the embodiments illustrated in FIGS. 3, 6 and 8, the bulb 140 comprises a base 150 and a globe 160, and the base 150 includes a lower section 152 and an upper section 154. The lower section 152 of the base 150 is smaller than the opening 130 in the blossom 120 and threads through the opening 130 and engages the socket 50. The upper section 154 of the base 150 is larger than the opening 130 of the blossom 120 and does not pass through the opening 130. The upper portion 154 of the base 150, therefore, abuts the blossom 120, securing the blossom 120 onto the stem 10 as the lower portion 152 of the base 150 engages the socket 50.

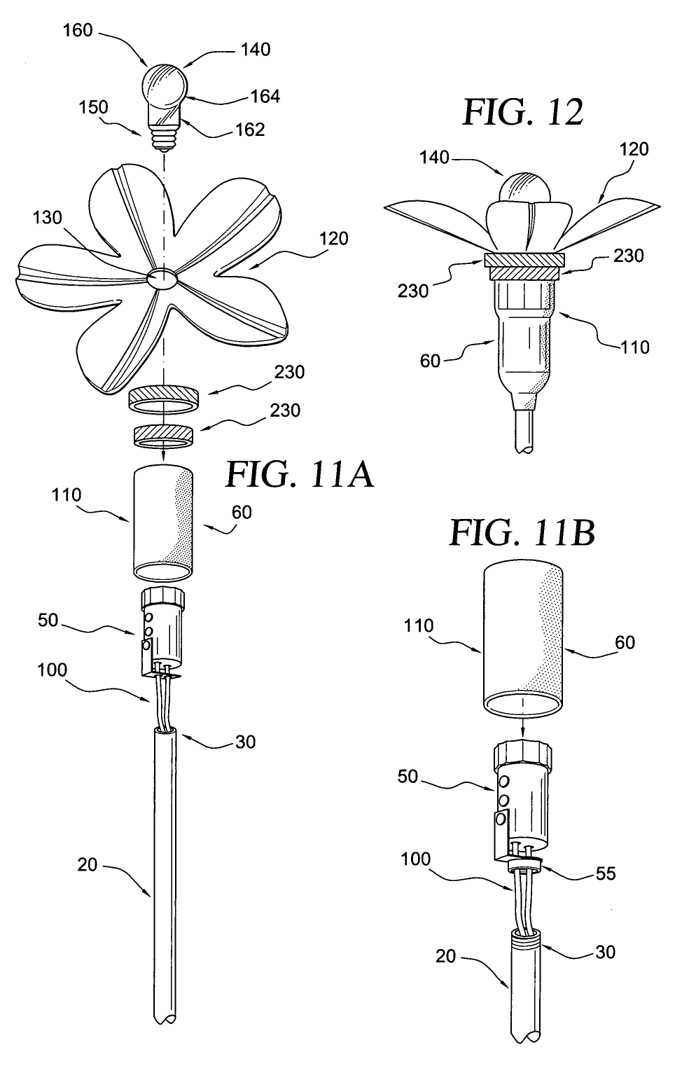

In another embodiment illustrated in FIG. 11A, the globe 160 of the bulb 140 includes a lower section 162 and an upper section 164. The base 150 and the lower section 162 of the globe 160 pass through the opening 130 of the blossom 120 and also pass through spacers 230 between the blossom 120 and the socket 50. The spacers 230 prevent electrical contact between the blossom 120 and the socket 50 and between the blossom 120 and the base 150 of the bulb 140. The upper section 164 of the globe 160 is larger than the opening 130 of the blossom 120 and does not pass through the opening 130. As the base 150 engages the socket 50, the upper portion 164 of the globe 160 abuts the blossom 120 and holds the blossom 120 onto the stem 10.

According to the invention, the collar 60 comprises a structure that protects, e.g., surrounds, the electrical socket 50. In one embodiment, illustrated in FIG. 3, the collar 60 comprises a common plumbing reducer fitting 70 having a cavity 80. In this embodiment, wires 100 of the socket 50 are associated with the shaft 20 by threading the wires 100 through the reducer fitting 70 and the upper end 30 of the shaft 20. The wires 100 exit through the lower end of the shaft 40, as illustrated in FIGS. 1 and 2. In this embodiment, the socket 50 is secured to the inside cavity 80 of the reducer fitting 70, for example, by hot glue.

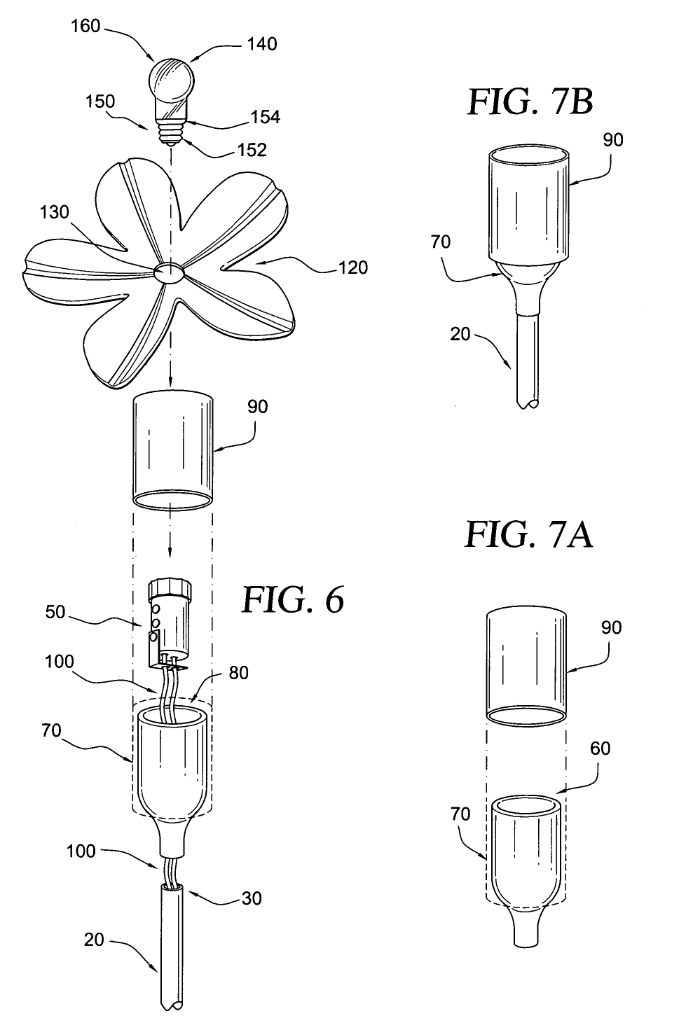

In a second embodiment, illustrated in FIGS. 6, 7A, and 7B, the collar 60 also includes a common plumbing coupling 90 which is slid over the reducer fitting 70 and socket 50. In this embodiment, the collar 60 is comprised of both the reducer fitting 70 and the coupling 90, as illustrated in FIG. 7A. In these embodiments, the end of the reducer fitting 70 opposite the socket 50 fits over the upper end 30 of the shaft 20. Of course, the coupling and reducer fitting can also be integrally formed.

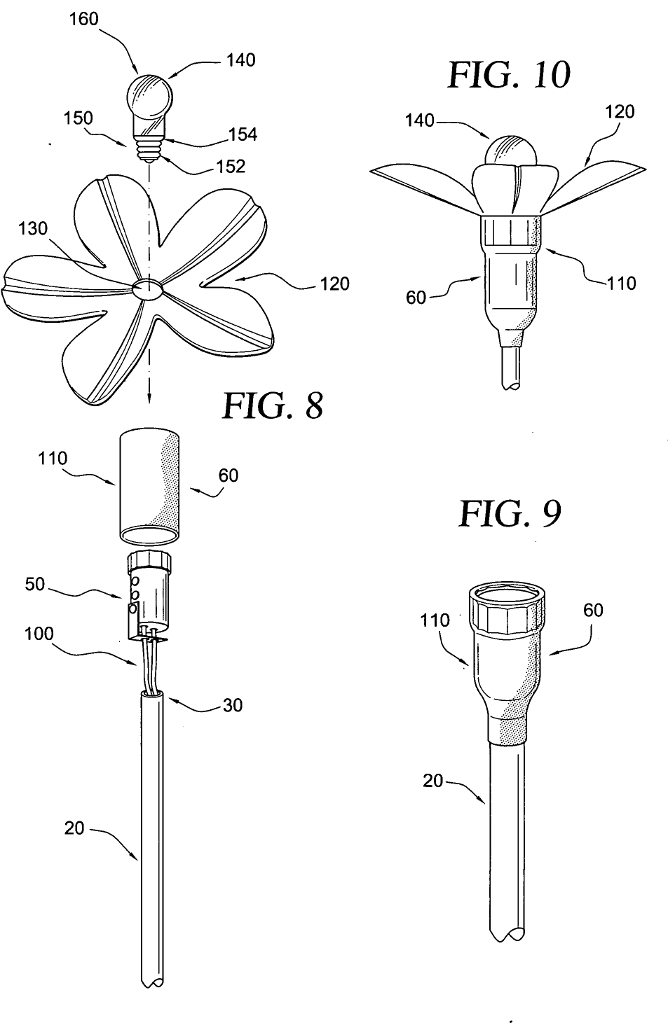

In yet another embodiment, illustrated in FIGS. 8, 9, 10, 11A, 11B, and 12, the collar 60 comprises a heat-shrinking sleeve 110. In these embodiments, the heat-shrinking sleeve 110 fits over the socket 50 and also the upper end 30 of the shaft 20.

The collar 60 is connected to the upper end 30 of the shaft 20 either separately or integrally. FIGS. 4, 7B, and 9 illustrate collars 60 formed separately and attached to the shaft 20. FIG. 5 illustrates a collar 60 formed integrally with the shaft 20.

In the embodiments illustrated in FIG. 3 and FIG. 6 the collar 60 is attached to the upper end 30 of the shaft 20. More particularly, adhesive may be applied to the collar 60, the upper end 30 or both and the collar 60 may be advanced over shaft 20, for example, using an adhesive and compression pressure by tapping the collar 60 with a rubber mallet. Because the socket 50 is secured to the inside cavity 80 of the reducer fitting 70, the socket 50 is thereby secured to the stem 10. FIG. 4 shows the assembled shaft 20 and reducer fitting 70. FIG. 7B shows the assembled shaft 20 and collar 60 illustrated in FIGS. 6 and 7A.

In the embodiment illustrated in FIG. 8 and 11A, the collar 60 is connected to the shaft 20 when the sleeve 110 is heat-shrunk, thereby securing the socket 50 to the shaft 20. In FIG. 11B, the socket 50 is secured both by the heat-shrunk sleeve 110 and also by tightening screw threads 55 on the socket 50 onto the shaft 20.

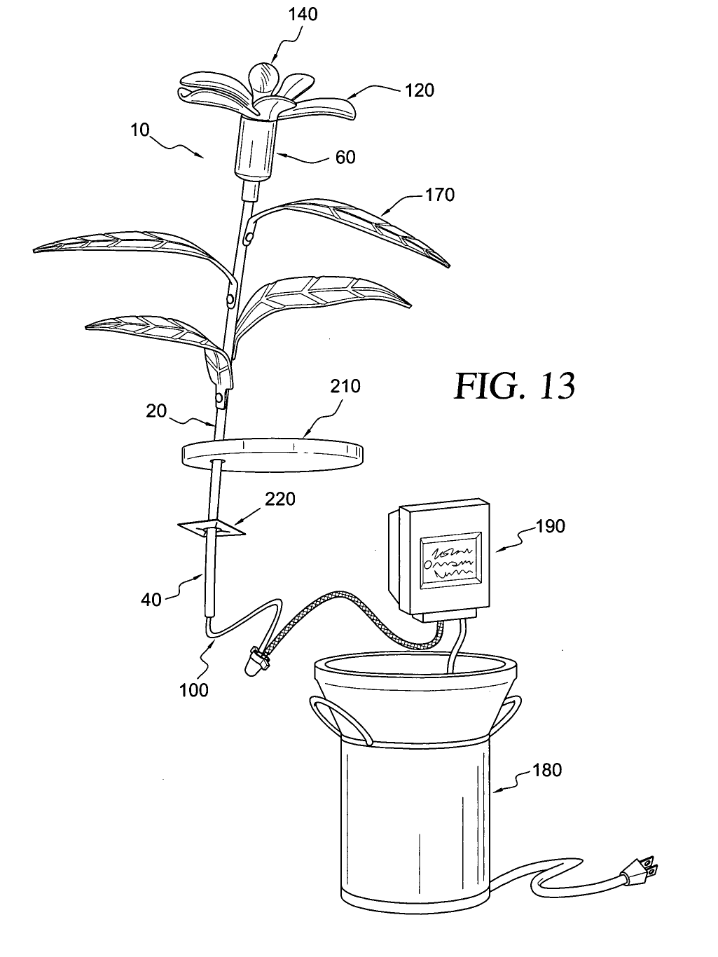

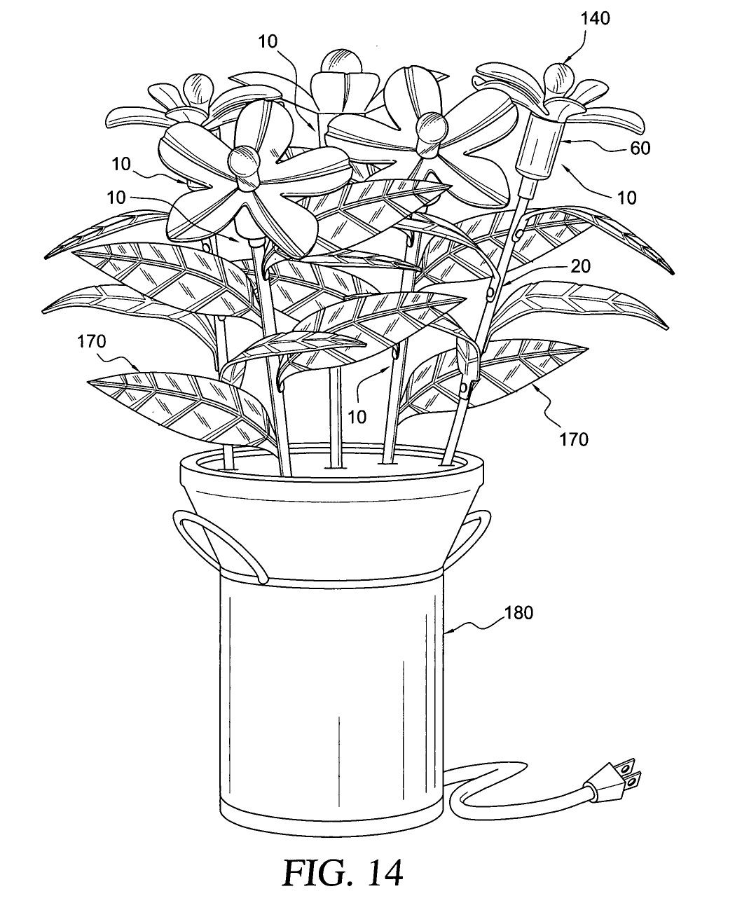

In a further embodiment the artificial flower assembly also comprises a container 180 in which a stem 10 is disposed. FIGS. 13 and 14 illustrate embodiments including a container 180. In the embodiment illustrated in FIG. 13, the container 180 includes a platform 210 made of a substantially rigid material such as STYROFOAM® or other rigid foam. The stem 10 is attached to the container 180 by inserting the lower end 40 of the shaft 20 through the platform 210 and attaching a retaining clip 220 onto the lower end 40 of the shaft 20. FIG. 14 illustrates several stems 10 in a bucket-shaped container 180.

In the embodiment illustrated in FIG. 13, the container 180 includes a transformer 190 which is electrically connected to the wires 100 of the stem 10. The transformer 190 preferably steps down power to the artificial flower assembly. This allows the same stem 10 and bulb 140 to be safely used both indoors and outdoors. In this embodiment, the transformer 190 is located inside the container 180, providing better protection for the electrical components and easier movement and placement of the entire flower assembly.

According to another embodiment of the invention, the artificial flower assembly includes a stake 200. In the embodiment illustrated in FIG. 15, the stake 200 is a generally rigid rod. One end of the stake 200 is inserted into the ground and the other end is inserted into the lower end 40 of the shaft 20 thereby supporting the flower assembly.

In another embodiment, illustrated in FIG. 16, the invention includes a timer 250 capable of automatically switching electrical current on and off. According to still another embodiment of the invention, the artificial flower assembly includes a solar power device 240, illustrated in FIG. 17. The solar power device 240 electrically charges from sunlight and thereafter provides electrical current to the flower assembly. The invention can also include a “dusk to dawn” light sensor, turning “on” at dusk and “off” at dawn. Yet a further embodiment of the invention includes a kit containing a plurality of stem assemblies, blossoms 120, bulbs 140, stakes 200, and a transformer 190. Each stem assembly includes a shaft 20 coupled to a socket 80. In the stem assembly, wires 100 are secured to or engaged with (either wound around or threaded through) the shaft 20. The socket 50 is surrounded by a collar 60 which is connected to the upper end 30 of the shaft 20. The kit may also include a container 180 and platform 210.

The invention also includes processes for manufacturing the artificial flower assembly. According to an embodiment, the process comprises the steps:
1) associating the wire 100 of the socket 50 with the shaft 20 of the stem 10,
2) surrounding the socket 50 with the collar 60, and
3) connecting the collar 60 to the upper end 30 of the shaft 20.

In at least one embodiment, the step of associating the wire 100 with the shaft 20 includes threading the wire 100 through the upper end 30 of the shaft 20 (see FIGS. 3, 6, 8, and 11A, 11B). In these embodiments, the wire 100 exits through the lower end 30 of the shaft 40 (see FIGS. 1, 2, 13, 15, 16, 17).

In an embodiment illustrated in FIG. 8, the steps of surrounding the socket 50 with the collar 60 and connecting the collar 60 to the upper end 30 of the shaft 20 include heat-shrinking a sleeve 110 onto the socket 50 and the upper end 30 of the shaft 20. In one embodiment, connecting the collar 60 to the shaft by heat shrinking secures the socket 50 to the shaft 20. (FIGS. 8, 11A). In another embodiment, illustrated in FIG. 11B, heat-shrinking the collar 60 works in combination with tightening screw threads 55 on the socket 50 to secure the socket 50 to the shaft 20.

In another embodiment, illustrated in FIG. 3, the process of surrounding the socket 50 with the collar 60 includes threading the wire 100 through a common plumbing reducer fitting 70 and securing the socket 50 to the cavity 80 of the reducer fitting 70 (for example, with hot glue). In another embodiment, illustrated in FIG. 6, the process of surrounding the socket 50 with the collar 60 also includes attaching a common plumbing coupling 90 onto the reducer fitting 70. In both of these embodiments, the step of connecting the collar 60 to the upper end 30 of the shaft 20 is accomplished by fitting the reducer fitting 70 over the upper end 30 of the shaft 20, for example using an adhesive and compression pressure of tapping the collar 60 with a rubber mallet.

In another embodiment, the manufacturing process includes attaching the stem 10 to a container 180. This process includes inserting the lower end 40 of the shaft 20 through a substantially rigid platform 210 (for example, STYROFOAM® or other rigid foam) and attaching a retaining device 220. The retaining device can be any apparatus capable of retaining the stem in the platform, for example, a clip attached to the lower end 40 of the shaft 20 (as illustrated in FIG. 13). In another embodiment, the retaining device may include a clip and plastic disc disposed on each side of platform 210. The plastic disk may include a single aperture to accommodate a single stem or may include multiple apertures to accommodate multiple stems. FIG. 13 also illustrates a further embodiment process in which a transformer 190 is connected to the wires 100 of the stem 10, and the transformer 190 is located inside the container 180.

In another embodiment, the process of manufacturing the artificial flower assembly includes coloring the blossoms 120 (for example, by spray-painting in a variety of colors and combinations).

The invention further includes processes for assembling the artificial flower assembly. According to an embodiment, the process comprises the steps:
1) threading the bulb 140 through the opening 130 in the removable blossom 120, and
2) engaging the bulb 140 with the socket 50, thereby securing the blossom 120 onto the stem 10.

In an embodiment, the process for assembling the artificial flower also includes applying a solution to the artificial flower assembly for enhancing oxidation (for example, when the artificial flower is made of copper).

In another embodiment, the assembly process includes engaging a stake 200 with the lower end 40 of the shaft 20 to support the flower assembly in the ground. In the embodiment illustrated in FIG. 15, the stake 200 is a generally rigid rod, and the assembly process comprises inserting one end of the stake 200 into the ground and inserting the other end of the stake 200 into the lower end 40 of the shaft 20.

In other embodiments, the assembly process includes electrically connecting the stem 10 to a transformer 190 (see FIG. 13) or to a solar power device 240 (FIG. 17) or to a timer device 250 (FIG. 16) which is capable of switching electrical power on and off on a timed basis.

Given the foregoing, it should be apparent that the specifically described embodiments are illustrative and not intended to be limiting. Furthermore, variations and modifications to the invention should now be apparent to a person having ordinary skill in the art. These variations and modifications are intended to fall within the scope and spirit of the invention as defined by the following claims.

I claim:

1. An artificial flower assembly comprising:
a stem including:
a flexible tubular shaft having an upper and lower end, an electrical socket, and
a collar surrounding said socket and connected to said upper end of said shaft; and
a removable blossom with an opening of a first diameter; and
a bulb being threaded through said blossom opening and engaged with said socket, a portion of said bulb abutting said blossom and securing said blossom onto said stem.

2. An artificial flower assembly as in claim 1 wherein said bulb includes a base comprising a lower section and an upper section, said lower section being threaded through said blossom opening and engaged with said socket and said upper section dimensionally sized larger than said first diameter and abutting and securing said blossom.

3. An artificial flower assembly as in claim 1 wherein said bulb includes a base and a globe, said globe comprising a lower section and an upper section, said base and said lower section of said globe being threaded through said blossom opening, said base being engaged with said socket, and said upper section of said globe dimensionally sized larger than said first diameter and abutting and securing said blossom; said flower assembly also including an annular spacer between said blossom and said collar adapted to prevent electrical contact between said blossom and said socket and between said blossom and said bulb base.

4. An artificial flower assembly as in claim 1 also including a leaf attached to said stem.

5. An artificial flower assembly as in claim 4 wherein at least one of said blossom and said leaf are made of aluminum.

6. An artificial flower assembly as in claim 4 wherein at least one of said blossom, said collar, said shaft, and said leaf are made of materials adapted to change appearance as a result of oxidation.

7. An artificial flower assembly as in claim 6 wherein at least one of said blossom, said collar, said shaft, and said leaf are made of copper.

8. An artificial flower assembly as in claim 4 wherein at least one of said blossom, said collar, said shaft, and said leaf are made of materials adapted to change appearance in response to changes in temperature.

9. An artificial flower assembly as in claim 4 wherein at least one of said blossom, said collar, said shaft, and said leaf are made of materials adapted to change appearance in response to moisture.

10. An artificial flower assembly as in claim 4 wherein at least one of said blossom, said collar, said shaft, and said leaf are made of phosphorescent materials.

11. An artificial flower assembly as in claim 1 further including a container comprising a compartment in which said stem is disposed.

12. An artificial flower assembly as in claim 11 further comprising a transformer disposed in said container and electrically connected to said stem.

13. An artificial flower assembly as in claim 1 including a stake associated with said lower end of said shaft and adapted to support said flower assembly.

14. An artificial flower assembly as in claim 13 wherein said stake is a generally rigid rod adapted to be inserted into said lower end of said shaft.

15. An artificial flower assembly as in claim 1 further comprising a transformer electrically connected to said stem.

16. An artificial flower assembly as in claim 1 further including a solar power device.

17. An artificial flower assembly as in claim 1 further including a timer for switching electrical current on and off on a timed basis.

18. An artificial flower assembly comprising:
a stem including:
a flexible tubular copper shaft having an upper end,
an electrical socket, and
a copper collar surrounding said socket and connected to said upper end of said shaft;
a copper leaf attached to said stem,
a removable copper blossom with an opening of a first diameter, the blossom including a plurality of petals;
a bulb including a base comprising a lower section and an upper section, said lower section being threaded through said blossom opening and engaged with said socket and said upper section dimensionally sized larger than said first diameter and abutting and securing said blossom onto said stem;
a container comprising a compartment in which said stem is disposed; and
a transformer disposed in said container and electrically connected to said stem.

19. An artificial flower assembly comprising:
a stem including:
a flexible tubular shaft having an upper end,
an electrical socket, and
a collar adapted to surround said socket and connect to said upper end of said shaft; and
a blossom with an opening of a first diameter; and
a bulb adapted to thread through said blossom opening and engage with said socket, a portion of said bulb adapted to abut said blossom and secure said blossom onto said stem.

20. An artificial flower assembly kit comprising:
a plurality of stems, each stem including:
a flexible tubular shaft having an upper end and a lower end,
an electrical socket engaged with said shaft, and
a collar surrounding said socket and connecting to the upper end of said shaft; and
a plurality of blossoms, each blossom having an opening of a first diameter;
a plurality of bulbs adapted to thread through said blossom opening and engage with said socket, a portion of said bulb adapted to abut said blossom and secure said blossom onto said stem; and
a plurality of stakes, each stake adapted to associate with said lower end of said shaft and supporting said flower assembly.

21. An artificial flower assembly kit as in claim 20 further comprising a transformer, wire, and electrical connectors.

22. A method for manufacturing an artificial flower assembly, the flower assembly comprising a stem including a flexible tubular shaft having an upper end and an lower end, an electrical socket including a wire, and a collar, said process comprising:
(a) associating said wire with said shaft,
(b) surrounding said socket with said collar, and
(c) connecting said collar to said upper end of said shaft.

23. A method for manufacturing an artificial flower assembly, as recited in claim 22, said collar comprising a heat-shrinking sleeve, wherein the steps of surrounding said socket with said collar and connecting said collar to said upper end of said shaft comprise heat-shrinking said sleeve onto said socket and said shaft.

24. A method for manufacturing an artificial flower assembly, as recited in claim 22, said collar comprising a reducer fitting having a cavity, wherein the step of surrounding said socket with said collar includes:
(a) threading said wire through said reducer fitting, and
(b) securing said socket to said cavity of said reducer fitting.

25. A method for manufacturing an artificial flower assembly, as recited in claim 24, said collar also comprising a coupling, wherein the step of surrounding said socket with said collar also includes attaching said coupling onto said reducer fitting.

26. A method for manufacturing an artificial flower assembly, as recited in claim 22, said artificial flower also comprising a removable blossom, said process further comprising coloring said blossom.

27. A method for manufacturing an artificial flower assembly, as recited in claim 22, said artificial flower assembly also including a container comprising a compartment, said process also including attaching said stem to said container.

28. A method for manufacturing an artificial flower assembly, as recited in claim 27, said container also including a substantially rigid platform and a retaining device, wherein the step of attaching said stem to said container comprises inserting the lower end of said shaft through said platform and attaching said retaining device.

29. A method for manufacturing an artificial flower assembly, as recited in claim 27, said artificial flower assembly also comprising a transformer, said process also including:
(a) locating said transformer inside said container, and
(b) electrically connecting said stem to said transformer.

30. A method for assembling an artificial flower assembly, the flower assembly comprising a stem including an electrical socket, a blossom with an opening of a first diameter, and a bulb, said process comprising:
(a) threading said bulb through said opening of said blossom, and
(b) engaging said bulb with said socket thereby securing said blossom onto said stem.

31. A method for assembling an artificial flower assembly, as recited in claim 30, said process also comprising applying to said artificial flower assembly a solution for enhancing oxidation.

32. A method for assembling an artificial flower assembly, as recited in claim 30, said artificial flower assembly also comprising a stake, said stem also comprising a flexible tubular shaft having a lower end, and said process also comprising engaging said stake with said lower end of said shaft to support said flower assembly.

33. A method for assembling an artificial flower assembly, as recited in claim 32 wherein said stake is a generally rigid rod, said step of associating said stake with said lower end of said shaft comprising inserting one end of said stake into said lower end of said shaft, and said process also comprising inserting the other end of said stake into the ground.

34. A method for assembling an artificial flower assembly, as recited in claim 30, said artificial flower assembly also comprising a transformer, said process also including electrically connecting said stem to said transformer.

35. A method for assembling an artificial flower assembly, as recited in claim 30 said artificial flower assembly also comprising a solar power device, said process also including electrically connecting said stem to said solar power device.

36. A method for assembling an artificial flower assembly, as recited in claim 30, said artificial flower assembly also comprising a timer device adapted to switch electrical power on and off on a timed basis, said process also including electrically connecting said stem to said timer device.
